(19) United States
(12) Patent Application Publication
(10) Pub. No.: US 2013/0289411 A1
(43) Pub. Date: Oct. 31, 2013

(54) APPARATUS TO REMOVABLY SECURE AN ULTRASOUND PROBE TO TISSUE

(71) Applicant: DBMEDX INC, Littleton, CO (US)

(72) Inventors: William L. Barnard, Maple Valley, WA (US); David B. Shine, Littleton, CO (US)

(73) Assignee: dBMEDx Inc, Littleton, CO (US)

(21) Appl. No.: 13/871,835

(22) Filed: Apr. 26, 2013

Related U.S. Application Data
(60) Provisional application No. 61/638,833, filed on Apr. 26, 2012.

(51) Int. Cl. A61B 8/00

(52) U.S. Cl. CPC A61B 8/42; A61B 8/4444
USPC 600/459

(57) ABSTRACT

A patch apparatus removably receives an ultrasound probe and attaches to a portion of a body, for example, via an adhesive. The patch apparatus may be disposable. The patch apparatus includes at least one cavity that sealingly holds a gel during use. The cavity may be sized to removably receive the ultrasound probe, in compression with the bodily tissue. Alternatively, the patch apparatus may include an adhesive or other fastener (e.g., hook and loop fastener) to removably retain the ultrasound probe. One or more magnets may indicate an orientation of the ultrasound probe or other information regarding an imaging procedure. An ultrasound probe may include a housing that is at least partially transparent, a display housed in the housing and visible from an exterior thereof, and one or more magnets to encode information regarding an orientation and/or procedure.

### BACKGROUND

1. Technical Field

This disclosure generally relates to monitoring of bodily or anatomical structures, and particularly to securing ultrasound transducers or other medical imaging devices to a portion of a body.

2. Description of the Related Art

Ultrasound imaging employs transducers to produce ultrasonic pressure waves and to detect return waves to perform imaging in a variety of environments. For example, ultrasound is effectively employed in medical imaging, allowing assessments of certain bodily tissue which would not otherwise be discernible without highly invasive techniques.

There are many commercially available ultrasound systems which provide images of bodily tissue, and even flow of bodily fluids, for example, blood flow.

A small three-dimensional (3D) imaging ultrasound system is described in U.S. patent application Ser. No. 12/948,622. An apparatus for ultrasound monitoring of anatomical features such as bladder volume over time is described in U.S. patent application Ser. No. 61/573,493, filed Sep. 6, 2011.

In order to obtain ultrasound images, an acoustic coupling medium is typically applied to the tissue of a subject (e.g., patient) and/or to an ultrasound transducer apparatus or scan head, referred to herein as a probe. The acoustic coupling medium may be applied as a liquid gel, for example, from a bottle. In order to obtain continuous ultrasound readings, a human operator typically manually holds a tethered ultrasound transducer apparatus, scan head or probe against the patient's skin. The operator continues holding the ultrasound transducer apparatus or scan head for as long as measurements need to be taken.

### BRIEF SUMMARY

A disposable patch selectively affixes a small, lightweight ultrasound device to a patient. Such facilitates ultrasound monitoring.

The disposable patch addresses any of three different issues related to autonomous, continuous ultrasound monitoring: 1) mechanical fixation; 2) ultrasonic coupling; and 3) ergonomic functionality. The disposable patch needs to physically hold the ultrasound scanner or probe against the body with sufficient pressure to create a reliable image. Practically speaking, the ultrasound scanner or probe must slightly deform the skin and subcutaneous fat tissue. However, the ultrasound scanner or probe must not deform tissue so much that the underlying anatomical structures being imaged are so perturbed as to create false readings. The ultrasound scanner or probe also must not apply so much pressure that skin necrosis results.

Acoustic coupling is required to get ultrasound energy into and back out of the human body. The acoustic coupling agent is typically quite viscous and messy. An ergonomic approach to deal with acoustic coupling medium (e.g., liquid gel), both application and removal thereof, would improve the entire monitoring process. This is especially useful in an emergency setting where space and time are at a premium.

A patch apparatus to removably secure an ultrasound probe to tissue may be summarized as including a substrate having a front face that faces tissue in use; an adhesive carried on the front face of the substrate; an ultrasound probe holder physically coupled to the substrate, the ultrasound probe holder having an ultrasound probe receiving cavity that removably receives the ultrasound probe at least partially therein; and a number of acoustic coupling medium reservoirs to releasably retain acoustic coupling medium, the acoustic coupling medium reservoirs fluidly communicatively coupled to selective dispense the acoustic coupling medium into the ultrasound probe receiving cavity.

The ultrasound probe holder may include a lid that at least partially forms the ultrasound probe receiving cavity. The lid may be pivotally coupled to pivot between an open position that provides access to the ultrasound probe receiving cavity from an exterior thereof, and a closed position in which the ultrasound probe receiving cavity is not accessible from the exterior thereof.

The patch apparatus may further include a number of elongated permanent magnets extending at least partially along a length of the ultrasound probe holder.

The ultrasound probe receiving cavity may be sized with at least one dimension smaller than a corresponding dimension of the ultrasound probe. The substrate may be a flexible foam substrate. The flexible foam substrate may have a through opening, the through opening in registration with the ultrasound probe receiving cavity. A portion of the ultrasound probe may extend through the through opening in the flexible foam substrate to extend partially beyond the front face. The acoustic coupling medium reservoirs may each be formed by a respective cavity in the flexible foam substrate. The acoustic coupling medium reservoirs may include a first and a second die cut cavity in the flexible foam substrate, the second die cut cavity opposed across the ultrasound probe receiving cavity from the first die cut cavity. The first and the second die cut cavities may each be fluidly coupled to the ultrasound probe receiving cavity by a respective set of tapered openings that form respective nozzles from which the acoustic coupling medium is selective dispensable under pressure exerted on the flexible foam substrate from an exterior thereof.

The patch apparatus may further include a selectively removable holding frame that seals the acoustic coupling medium reservoirs from the ultrasound probe receiving cavity until selectively removed.

The patch apparatus may further include a first elongated permanent magnet extending at least partially along a length of the ultrasound probe holder; and a second elongated permanent magnet extending at least partially along the length of the ultrasound probe holder and opposed across the ultrasound probe receiving cavity from the first elongated permanent magnet.

The ultrasound probe holder may include an adhesive. The adhesive may be a biocompatible adhesive carried on the front face of the substrate. The adhesive may be a biocompatible pressure sensitive adhesive carried on a microporous tape on the front face of the substrate, and may further include a selectively removable release liner that covers the pressure sensitive adhesive prior to use.

The patch apparatus may further include an adhesive that adhesively secures the ultrasound probe holder to the substrate.

The adhesive may adhesively secure the ultrasound probe holder to a back face of the substrate, the back face opposed to the front face across a thickness of the substrate.

A patch apparatus to removably secure an ultrasound probe to tissue may be summarized as including a substrate having a front face that faces tissue in use, a back face that is opposed from the front face across a thickness of the substrate, the substrate that forms an acoustic coupling medium reservoir with a front opening that extends through the front face of the substrate and a back opening that extends through the back face of the substrate; an adhesive carried on the front face of the substrate to selectively detachably couple the substrate to tissue; a front cover positioned between the acoustic coupling medium reservoir and the front opening to seal the acoustic coupling medium reservoir from an exterior thereof, and selectively operable to provide fluid communication between the acoustic coupling medium reservoir and tissue to which the substrate is physically coupled in use; and a back cover positioned between the acoustic coupling medium reservoir and the back opening to seal the acoustic coupling medium reservoir from the exterior thereof, and selectively operable to provide fluid communication between the acoustic coupling medium reservoir and an ultrasound probe which is physically coupled to the substrate in use.

The substrate may includes a first slot and at least a second slot, the front cover may include a front cover tab that extends at least partially through the first slot to allow selective at least partial withdrawal of the front cover from a respective initial position to provide fluid communication between the acoustic coupling medium reservoir and tissue to which the substrate is physically coupled in use, and the back cover may include a back cover tab that extends at least partially through the second slot to allow selective at least partial withdrawal of the back cover from a respective initial position to provide fluid communication between the acoustic coupling medium reservoir and an ultrasound probe which is physically coupled to the substrate in use.

The first slot and at least a second slot may be self-sealing slots. The front cover and the back cover may each be perforated plastic covers.

The patch apparatus may further include the ultrasound probe, wherein the ultrasound probe has a housing including at least one of a flat, tab or peripheral flange to sealingly physically couple with a portion of the substrate.

A ultrasound probe may be summarized as including a housing having an interior, at least one wall that separates the interior from an exterior thereof, and at least one window; a display rotatably mounted in the housing, the display visible from the exterior via the window; and at least one sensor positioned in the housing, the at least one sensor responsive to a magnetic field produced by a number of magnets carried by a patch apparatus to which the ultrasound probe is removably physically coupled in use.

The at least one window may include an at least partially transparent portion of the at least one wall of the housing, and the display may include a plurality of light emitting diodes carried by at least one substrate rotatably mounted in the housing.

### BRIEF DESCRIPTION OF THE SEVERAL VIEWS OF THE DRAWINGS

In the drawings, identical reference numbers identify similar elements or acts. The sizes and relative positions of elements in the drawings are not necessarily drawn to scale. For example, the shapes of various elements and angles are not drawn to scale, and some of these elements are arbitrarily enlarged and positioned to improve drawing legibility. Further, the particular shapes of the elements as drawn, are not intended to convey any information regarding the actual shape of the particular elements, and have been solely selected for ease of recognition in the drawings.

FIG. 1 is a side elevational view of a disposable patch apparatus and a three-dimensional (3D) ultrasound probe removably retained by a portion of the disposable patch apparatus, according to one illustrated embodiment.

FIG. 2 is a side elevational view of the disposable patch apparatus of FIG. 1 with the 3D ultrasound probe removed therefrom and a holding frame in place.

FIG. 3 is a bottom plan view of the disposable patch apparatus of FIG. 1 and the 3D ultrasound probe retained thereby, illustrating a number of acoustic coupling medium liquid gel squeeze pouches of the disposable patch apparatus.

FIG. 4 is a bottom, side elevational view of a 3D ultrasound scanner probe that may be used with various embodiments described herein, the 3D ultrasound scanner probe a housing with a flat, tab or flange, according to one illustrated embodiment.

FIG. 5 is a front, side, bottom elevational view of the 3D ultrasound scanner probe of FIG. 4.

FIG. 6 is a front elevational view of a disposable patch apparatus with front and back covers in place, according to another illustrated embodiment

FIG. 7 is an exploded view of the disposable patch apparatus of FIG. 6 with front and back covers spaced away from a main portion thereof.

FIG. 8 is a cutaway view of the disposable patch apparatus of FIGS. 6 and 7 with a 3D ultrasound scanner probe attached thereto.

### DETAILED DESCRIPTION

In the following description, certain specific details are set forth in order to provide a thorough understanding of various disclosed embodiments. However, one skilled in the relevant art will recognize that embodiments may be practiced without one or more of these specific details, or with other methods, components, materials, etc. In other instances, well-known structures associated with ultrasound systems and transducers have not been shown or described in detail to avoid unnecessarily obscuring descriptions of the embodiments.

Unless the context requires otherwise, throughout the specification and claims which follow, the word “comprise” and variations thereof, such as, “comprises” and “comprising” are to be construed in an open, inclusive sense, that is as “including, but not limited to.”

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

The headings and Abstract of the Disclosure provided herein are for convenience only and do not interpret the scope or meaning of the embodiments.

FIGS. 1-3 show a disposable patch apparatus 100 to removably hold a three-dimensional (3D) ultrasound scanner probe 102, according to one illustrated embodiment. The disposable patch apparatus 100 may advantageously achieve mechanical fixation of the 3D ultrasound scanner probe 102 to tissue (e.g., bodily tissue, for instance, skin). The disposable patch apparatus 100 may advantageously achieve ultrasonic coupling between the 3D ultrasound scanner probe 102 and the tissue. The disposable patch apparatus 100 may advantageously achieve ergonomic functionality in medical imaging of tissue.

The disposable patch apparatus 100 includes a substrate 104 and an ultrasound probe holder 106 that forms an ultrasound probe receiving cavity 108 to removably hold the 3D ultrasound scanner probe 102 and to hold acoustic coupling media 110.

The substrate 104 includes a front face 104a that in use faces tissue to be imaged. The substrate 104 may include a back face 104b that is opposed from the front face 104a across a thickness 104c of the substrate 104. The substrate 104 may, for example, take the form of a flexible foam substrate (e.g., open or closed cell polyurethane foams). The flexible foam substrate 104 may include at least one through opening, hole, or aperture 114, sized and dimensioned to allow a portion of the 3D ultrasound scanner probe 102 to extend therethrough, past the front face 104a of the substrate 104.

As best illustrated in FIG. 3, the substrate 104 may include a number of acoustic coupling medium reservoirs (two shown) 116a, 116b (collectively 116) to releasably retain acoustic coupling medium 110. The acoustic coupling medium reservoirs 116 are fluidly communicatively coupled to selectively dispense the acoustic coupling medium 110 into the ultrasound probe receiving cavity 108. The acoustic coupling medium reservoirs 116 may, for example, take the form of one or more cavities or pockets in the flexible foam substrate 104. The cavities or pockets may be fluidly communicatively coupled by a number of tapered, narrowed or restricted passages 118a, 118n (eight shown, only two called out for clarity of illustration, collectively 118) in the flexible foam substrate 104 which form respective nozzles. Preferably, there are a plurality of nozzles for each of the acoustic coupling medium reservoirs 116 (e.g., four illustrated for each acoustic coupling medium reservoir), the nozzles spaced along a dimension of the substrate 104 to facilitate distribution of acoustic coupling media 110 throughout the ultrasound probe receiving cavity 108. The acoustic coupling medium reservoirs 116 and/or the passages or nozzles 118 may be formed by die cutting the flexible foam substrate 104. The acoustic coupling medium reservoirs 116 may hold and/or dispense measured amounts of liquid gel acoustic coupling media 110 into the ultrasound probe receiving cavity 108.

The front face 104a of the substrate 104 may carry an adhesive 120 suitable for detachably physically coupling the disposable patch apparatus 100 to the tissue. The adhesive 120 may be carried by the front face 104a directly or indirectly, for example, carried on or impregnated in a microporous tape 122. The microporous tape 122 may be double sided, allowing the microporous tape 122 to be adhesively secured to the front face 104a of the substrate 104 while also being selectively adhesively secured to tissue. The adhesive 120 may, for instance, be a pressure sensitive adhesive. In such instances, the disposable patch apparatus 100 may include a selectively removable release liner 124, which is removed in use to expose the pressure sensitive adhesive 120. The adhesive 120 is preferably bio-compatible, preventing or reducing the possibility of reactions with the tissue (e.g., human skin).

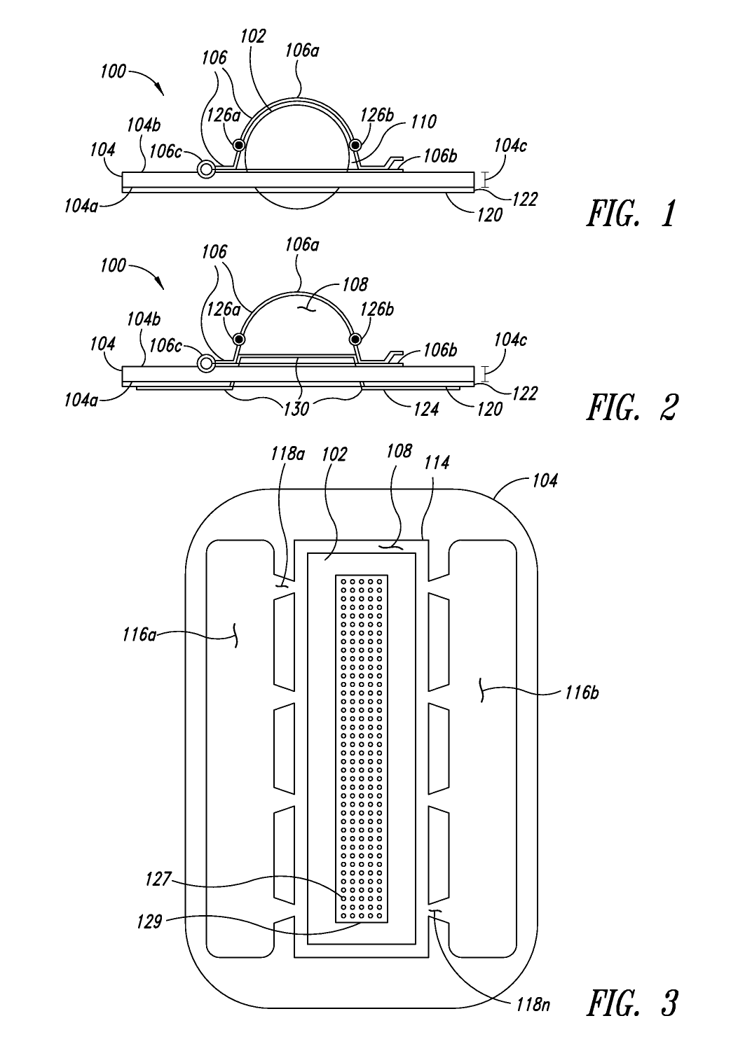

The ultrasound probe holder 106 that forms an ultrasound probe receiving cavity 108 to removably hold the 3D ultrasound scanner probe 102 may take the form of an enclosure structure. The ultrasound probe holder 106 may, for instance, be formed of a plastic, for example, polyethylene teraphthalate (PET). The ultrasound probe holder 106 may include a base 106a and a lid 106b, the base 106a securely physically coupled or attached to the back face 104b of the substrate 104. The lid 106b may be moveably coupled to the base 106, for example, via a hinge, joint or flexure 106c. The lid 106b is movable between an open position (not illustrated) and a closed position (illustrated in FIGS. 1 and 2). In the open position, the lid 106b is moved (e.g., pivoted) to allow access to the ultrasound probe receiving cavity 108 from an exterior thereof, permitting placement of an ultrasound probe 102 at least partially therein. In the closed position, the lid 106b physically secures or holds the ultrasound probe 102 in the ultrasound probe receiving cavity 108, for instance, pressed against the substrate 104.

In the closed position, the lid 106b seals to the base 106a to contain acoustic coupling media 110 (e.g., liquid gel) from the back face 104b of the disposable patch apparatus 100. The lid 106b has a mechanism or structure that seals to a major portion of the base 106a or substrate 104. For example, an adhesive or tactifier may allow the lid 106b to seal to a major portion of the base 106a or substrate 104 of the disposable patch apparatus 100. Such may be similar in some respects to that employed with packaging commonly associated with prepackaged deli meat.

The hinge or joint or flexure 106c allow the lid 106b to be opened to receive the 3D ultrasound scanner probe 102 and then closed after the 3D ultrasound scanner probe 102 is inserted in a scanner probe receiving cavity 108 formed by the lid 106b and/or major portion of the disposable patch apparatus 100. The scanner probe receiving cavity 108 has a dimension (e.g., diameter) that is smaller than a corresponding dimension (e.g., diameter) of the 3D ultrasound scanner probe 102, so that in use the 3D ultrasound scanner probe 102 is pressed into a patient's tissue.

As best illustrated in FIGS. 1 and 2, the lid 106b may also incorporate one or more (e.g., two) permanent magnets 126a, 126b (two shown, collectively 126) which are optionally used by the 3D ultrasound scanner probe 102 to locate where a display 127 should be positioned. The permanent magnets 126 may be simple inexpensive permanent magnets. The display 127 is created by selectively illuminating a line of LEDs as they spin 360° inside a housing of the 3D ultrasound scanner probe 102, which has at least one visibly translucent wall or window 129. A sensor (e.g., Hall effect sensor) in the 3D ultrasound scanner probe 102 detects the permanent magnets 126 and can orient the display to be between the two magnets in rotational space.

The permanent magnets 126 may be located at a mid-point along a length of the scanner probe receiving cavity 108 so that the 3D ultrasound scanner probe 102 can be placed in the scanner probe receiving cavity 108 in either orientation (e.g., either end of the scanner probe proximate a respective end of the scanner probe receiving cavity 108). The permanent magnets 126 may further be located roughly 120° apart so that a processor or other microcontroller (not illustrated) can distinguish orientation (e.g., between ‘up’ and ‘down’) based on sensed positions or orientations of the permanent magnets 126. Additionally, the relative orientation (e.g., relative orientation of poles) of the permanent magnets 126 may be used to identify a particular type of procedure which is to be, is being, or has been performed. For example, the presence of the permanent magnets 126 at a 120° separation may indicate that the 3D ultrasound scanner probe 102 is being used in an examination of an inferior vena cava (IVC)/aorta or in a monitoring function or examination of such bodily tissue. This allows the same scanner hardware to also function as a different type of monitor. For example, sensing magnets that are spaced 90° apart may indicate use in a bladder monitor operation. Other arrangements of permanent magnets 126 could be achieved, for example if a handle were snapped on a cylinder of the 3D ultrasound scanner probe 102 with permanent magnets 126 spaced at, for example, 60° apart, or 30° apart, or even non-existent.

As noted above, the disposable patch apparatus 100 may include a number (e.g., two) of acoustic coupling medium reservoirs 116 that releasably or dispensably hold an acoustic coupling medium 110. Acoustic coupling media, for instance, in the form of liquid gel, may be pre-loaded into one or more acoustic coupling medium reservoirs 116, and be selectively dispensable therefrom. For example, a number of squeezable cavities or pockets may be formed with or in a die-cut foam substrate 104. The liquid gel cavities or pockets may have multiple injection ports or nozzles 118, from which the liquid gel may be dispensed (e.g., squished) into the scanner probe receiving cavity 108, for example, by application of pressure to the substrate 104. These ports or nozzles 118 may be blocked during storage and shipment to prevent unintentional dispensing of the acoustic coupling media held therein. For example, a portion of a holding frame 130 that serves to hold the disposable patch apparatus 100, may block the ports or nozzles 118. The holding frame 130 may, for example, take the form of a PET molded frame. Removal of the patch apparatus 100 and/or holding frame 130 may open the ports or nozzles 118, to allow dispensing of the acoustic coupling media 110 to the ultrasound probe receiving cavity 108 from the acoustic coupling medium reservoirs 116. In some implementations, a portion of the holding frame 130 may serve as the release liner, overlying the pressure sensitive adhesive 120 until the holding frame 130 is removed.

The acoustic coupling medium reservoirs 116 may each hold the same type of acoustic coupling medium 110, or different acoustic coupling media 110 may be stored in respective ones of the acoustic coupling medium reservoirs 116. Such may advantageously allow an operator to easily chose from a variety of acoustic coupling media 110, and cleanly dispense a selected or desired coupling medium 110.

The disposable patch apparatus 100 may have, or may carry, a bio-compatible (e.g., skin-friendly) micro-porous adhesive to secure and hold the disposable patch apparatus 100 to a patient's body. Such adhesive may be similar to adhesives commonly used with EKG leads. Such may be in the form of one or more adhesive pads.

The entire disposable patch apparatus 100 may typically be shipped in a standard medical pouch that can be sterilized using Ethylene Oxide or an equivalent modality.

In a typical method of using the patch assembly 100, the user initially peels a top layer of pouch off of holding frame 130. The user then removes the patch 100 from holding frame 130. The removal of the holding frame 130 opens the acoustic medium (e.g., gel) injector ports or nozzles 118. The user then presses the disposable patch apparatus on the patient in a desired location on the patient or subject. The user pushes down on substrate 104 to dispense acoustic coupling media 110 (e.g., liquid gel), for instance, squeezing the liquid gel into ultrasound probe receiving cavity 108.

The user may then lift the lid 106b to gain access to the ultrasound probe receiving cavity 102. The user may insert 3D ultrasound scanner probe 102 into ultrasound probe receiving cavity 108. The user then closes the lid 106b. The 3D ultrasound scanner probe 102 may be securely retained between the substrate 104 and the lid 106b, a portion of the 3D ultrasound scanner probe 102 extending through the substrate 104 beyond the front face 104a thereof.

If the acoustic coupling medium 110 gets absorbed over time, the user may squeeze more acoustic coupling medium 110 into the ultrasound probe receiving cavity 108. The user may palpate the entire disposable patch apparatus 100 to move the acoustic coupling medium 110 under the 3D ultrasound scanner probe 102. This can be performed with little or no mess. The 3D ultrasound scanner probe 102 may be retrieved from the disposable patch apparatus 100, for example, after conclusion of a diagnostic procedure, for reuse. The disposable patch apparatus 100 may then be disposed of, while the relatively more expensive 3D ultrasound scanner probe 102 is saved for reuse.

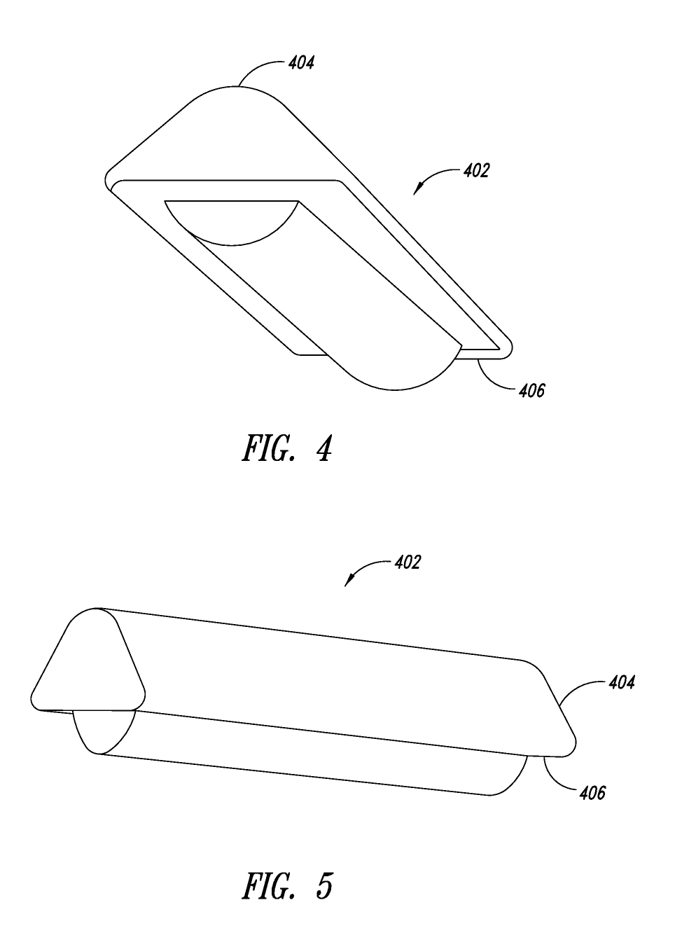

FIGS. 4 and 5 show a 3D ultrasound scanner probe 402, according to another illustrated embodiment. The 3D ultrasound scanner probe 402 may include a scanner housing 404 having one or more flats, tabs or peripheral flanges 406. The flats, tabs or peripheral flanges 406 may allow mounting or secure coupling of the scanner housing 404 to a disposable patch apparatus (e.g., disposable patch apparatus 100), for instance, via an adhesive (e.g., pressure sensitive adhesive). Such contrasts with the cylindrical housing of the 3D ultrasound scanner probe 102 are illustrated in FIGS. 1 and 3.

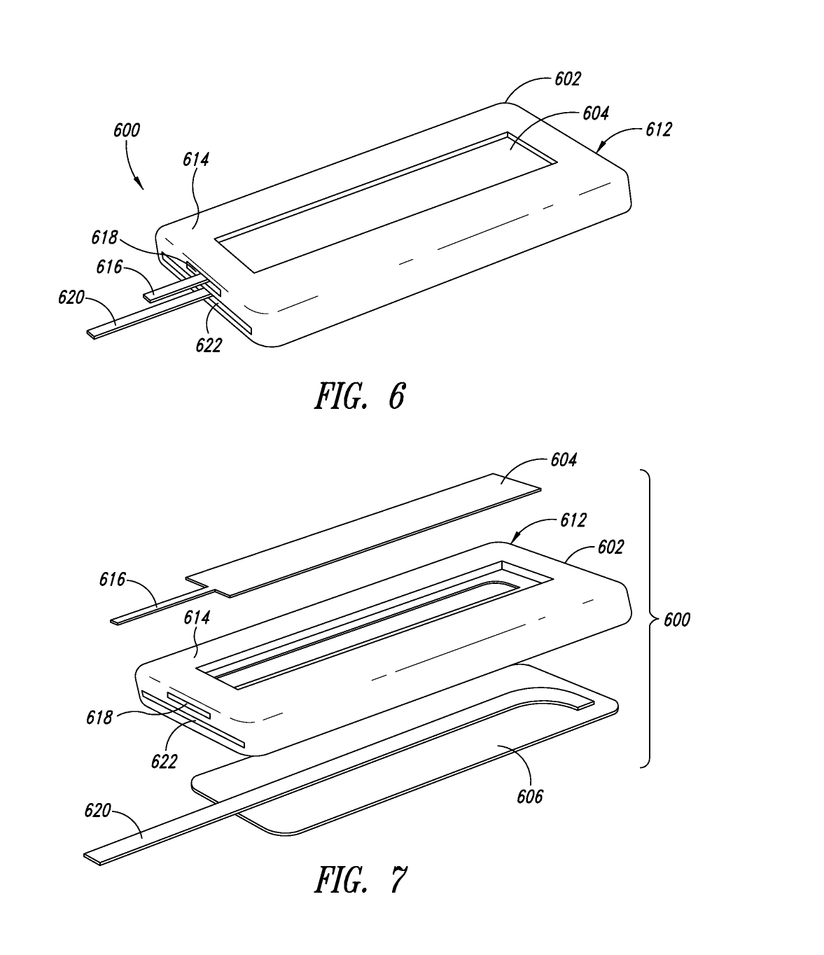

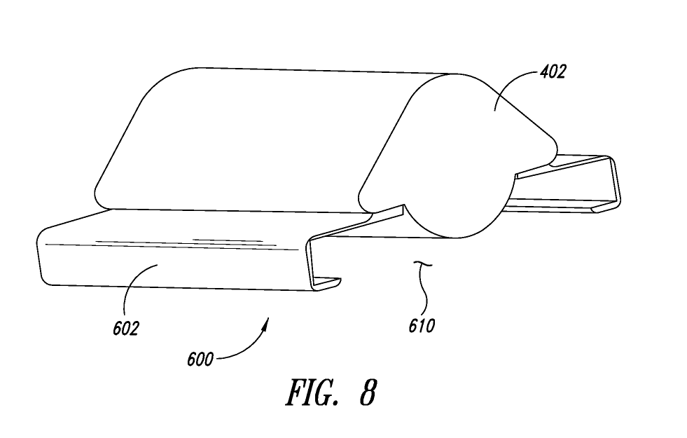

FIGS. 6-8 show a disposable patch apparatus 600, according to another illustrated embodiment. In particular, FIG. 6 shows the disposable patch apparatus 600 with a main housing 602, a front cover 604 and a back cover 606 in place to seal acoustic coupling medium therein prior to use. FIG. 7 shows the disposable patch apparatus 600 with the front cover 604 and back cover 606 spaced therefrom. FIG. 8 shows a cut-away view of a 3D ultrasound scanner probe 402 attached to the disposable patch apparatus 600 with the front and back covers 604, 606 removed from the main housing 602. The front and back covers 604, 606 may, for example, take the form of plastic covers. Notably, acoustic coupling media is not illustrated in FIGS. 7-8, but would fill the cavity 610 (FIG. 8) in the main housing 602 disposable patch apparatus 600.

The disposable patch apparatus 600 may include an adhesive pad (not visible) on a top of the main housing 602 to removably adhere the 3D ultrasound scanner probe 402 to the main housing 602. Such may be in lieu of locating the 3D ultrasound scanner probe 402 in a scanner probe receiving cavity (e.g., ultrasound probe receiving cavity 108 of the disposable patch apparatus 100 of FIG. 1). Acoustic coupling media (e.g., liquid gel) may be pre-loaded into a central cavity 610 of the main housing 602 of the disposable patch apparatus 600. The perforated, removable plastic sheets 604, 606 cover the two sides of the main housing 602 until use, retaining the acoustic coupling media therein.

In a typical method of using the patch assembly 600, the user initially adheres the 3D ultrasound scanner probe 402 to a back face of the main housing 602 of the disposable patch apparatus 600, forming a tight seal around the entire perimeter of the 3D ultrasound scanner probe 402. The user may adhere the front face 612 of the disposable patch apparatus 600 to the skin of patient, forming a tight, comfortable seal, for example, via an adhesive pad 614 carried on the front face.

The user may pull a front cover tab 616 to remove or at least displace the perforated plastic front cover 604 in order to expose the tissue (e.g., skin) to the acoustic coupling media. The front cover tab 616 may extend through a small slit 618 in the main housing 602 to be accessible from an exterior thereof. The slit 618 may be self-sealing, for instance, including compliant, elastic and/or resilient lips. Such may also serve to release trapped air.

The user may pull the back cover tab 620 to remove or at least partially displace the perforated plastic back cover 606 to expose the 3D ultrasound scanner probe 402 to the acoustic coupling media. The back cover tab 620 may extend through another small slit 622 in the main housing 602 to be accessible from an exterior thereof. The slit 622 may be self-sealing, for instance, including compliant, elastic and/or resilient lips. In some implementations, the front and the back cover tabs 616, 620 may both extend through a single, common slit.

In some embodiments, a disposable kit may be provided. The disposable kit may, for example, include one 3D ultrasound scanner probe 102, 402, and a plurality of disposable patch apparatus 100, 600. The disposable patch apparatus 100, 600 could also have one or more valves, for example, placed around a perimeter thereof, to allow additional acoustic coupling media to be dispensed from a separate container of acoustic coupling media. The separate container of acoustic coupling media may be supplied as part of, or along with, the disposable patch apparatus 100, 600 as part of the disposable kit.

The methods illustrated and described herein may include additional acts and/or may omit some acts. The methods illustrated and described herein may perform the acts in a different order. Some of the acts may be performed sequentially, while some acts may be performed concurrently with other acts. Some acts may be merged into a single act through the use of appropriate circuitry.

The various embodiments described above can be combined to provide further embodiments.

To the extent that they are not inconsistent with the teachings herein, the teachings of: U.S. patent application Ser. No. 12/948,622, filed Nov. 17, 2010; U.S. provisional patent application Ser. No. 61/573,493, filed Sep. 6, 2011; U.S. provisional patent application Ser. No. 61/621,877, filed Apr. 9, 2012; and U.S. provisional patent application Ser. No. 61/638,925, filed Apr. 26, 2012; U.S. provisional patent application Ser. No. 61/638,833, filed Apr. 26, 2012, and U.S. nonprovisional patent application Ser. No. ______, filed Apr. 26, 2013 in the names of William L. Barnard and David Bartholomew Shine and entitled “ULTRASOUND APPARATUS AND METHODS TO MONITOR BODILY VESSELS”, are each incorporated herein by reference in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A patch apparatus to removably secure an ultrasound probe to tissue, the patch apparatus comprising:
a substrate having a front face that faces tissue in use;
an adhesive carried on the front face of the substrate;
an ultrasound probe holder physically coupled to the substrate, the ultrasound probe holder having an ultrasound probe receiving cavity that removably receives the ultrasound probe at least partially therein; and
a number of acoustic coupling medium reservoirs to releasably retain acoustic coupling medium, the acoustic coupling medium reservoirs fluidly communicatively coupled to selective dispense the acoustic coupling medium into the ultrasound probe receiving cavity.

2. The patch apparatus of claim 1 wherein the ultrasound probe holder comprises a lid that at least partially forms the ultrasound probe receiving cavity.

3. The patch apparatus of claim 2 wherein the lid is pivotally coupled to pivot between an open position that provides access to the ultrasound probe receiving cavity from an exterior thereof, and a closed position in which the ultrasound probe receiving cavity is not accessible from the exterior thereof.

4. The patch apparatus of claim 3, further comprising:
a number of elongated permanent magnets extending at least partially along a length of the ultrasound probe holder.

5. The patch apparatus of claim 2 wherein the ultrasound probe receiving cavity is sized with at least one dimension smaller than a corresponding dimension of the ultrasound probe.

6. The patch apparatus of claim 1 wherein the substrate is a flexible foam substrate.

7. The patch apparatus of claim 6 wherein the flexible foam substrate has a through opening, the through opening in registration with the ultrasound probe receiving cavity.

8. The patch apparatus of claim 7 wherein a portion of the ultrasound probe extends through the through opening in the flexible foam substrate to extend partially beyond the front face.

9. The patch apparatus of claim 6 wherein the acoustic coupling medium reservoirs are each formed by a respective cavity in the flexible foam substrate.

10. The patch apparatus of claim 6 wherein the acoustic coupling medium reservoirs includes a first and a second die cut cavity in the flexible foam substrate, the second die cut cavity opposed across the ultrasound probe receiving cavity from the first die cut cavity.

11. The patch apparatus of claim 10 wherein the first and the second die cut cavities are each fluidly coupled to the ultrasound probe receiving cavity by a respective set of tapered openings that form respective nozzles from which the acoustic coupling medium is selective dispensable under pressure exerted on the flexible foam substrate from an exterior thereof.

12. The patch apparatus of claim 1, further comprising:
a selectively removable holding frame that seals the acoustic coupling medium reservoirs from the ultrasound probe receiving cavity until selectively removed.

13. The patch apparatus of claim 1, further comprising:
a first elongated permanent magnet extending at least partially along a length of the ultrasound probe holder; and
a second elongated permanent magnet extending at least partially along the length of the ultrasound probe holder and opposed across the ultrasound probe receiving cavity from the first elongated permanent magnet.

14. The patch apparatus of claim 1 wherein the ultrasound probe holder comprises an adhesive.

15. The patch apparatus of claim 14 wherein the adhesive is a biocompatible adhesive carried on the front face of the substrate.

16. The patch apparatus of claim 14 wherein the adhesive is a biocompatible pressure sensitive adhesive carried on a microporous tape on the front face of the substrate, and further comprising:
a selectively removable release liner that covers the pressure sensitive adhesive prior to use.

17. The patch apparatus of claim 1, further comprising:
an adhesive that adhesively secures the ultrasound probe holder to the substrate.

18. The patch apparatus of claim 17 wherein the adhesive adhesively secures the ultrasound probe holder to a back face of the substrate, the back face opposed to the front face across a thickness of the substrate.

19. A patch apparatus to removably secure an ultrasound probe to tissue, the patch apparatus comprising:
a substrate having a front face that faces tissue in use, a back face that is opposed from the front face across a thickness of the substrate, the substrate which forms an acoustic coupling medium reservoir with a front opening that extends through the front face of the substrate and a back opening that extends through the back face of the substrate;
an adhesive carried on the front face of the substrate to selectively detachably couple the substrate to tissue;
a front cover positioned between the acoustic coupling medium reservoir and the front opening to seal the acoustic coupling medium reservoir from an exterior thereof, and selectively operable to provide fluid communication between the acoustic coupling medium reservoir and tissue to which the substrate is physically coupled in use; and
a back cover positioned between the acoustic coupling medium reservoir and the back opening to seal the acoustic coupling medium reservoir from the exterior thereof, and selectively operable to provide fluid communication between the acoustic coupling medium reservoir and an ultrasound probe which is physically coupled to the substrate in use.

20. The patch apparatus of claim 19 wherein the substrate includes a first slot and at least a second slot, the front cover includes a front cover tab that extends at least partially through the first slot to allow selective at least partial withdrawal of the front cover from a respective initial position to provide fluid communication between the acoustic coupling medium reservoir and tissue to which the substrate is physically coupled in use, and the back cover includes a back cover tab that extends at least partially through the second slot to allow selective at least partial withdrawal of the back cover from a respective initial position to provide fluid communication between the acoustic coupling medium reservoir and an ultrasound probe which is physically coupled to the substrate in use.

21. The patch apparatus of claim 20 wherein the first slot and at least a second slot are self-sealing slots.

22. The patch apparatus of claim 19 wherein the front cover and the back cover are each perforated plastic covers.

23. The patch apparatus of claim 19, further comprising:
the ultrasound probe, wherein the ultrasound probe has a housing including at least one of a flat, tab or peripheral flange to sealingly physically couple with a portion of the substrate.

24. A ultrasound probe, the ultrasound probe comprising:
a housing having an interior, at least one wall that separates the interior from an exterior thereof, and at least one window;
a display rotatably mounted in the housing, the display visible from the exterior via the window; and
at least one sensor positioned in the housing, the at least one sensor responsive to a magnetic field produced by a number of magnets carried by a patch apparatus to which the ultrasound probe is removably physically coupled in use.

25. The ultrasound probe of claim 24 wherein the at least one window includes an at least partially transparent portion of the at least one wall of the housing, and the display includes a plurality of light emitting diodes carried by at least one substrate rotatably mounted in the housing.
